(19) United States
(12) Patent Application Publication
(10) Pub. No.: US 2006/0073184 A1
(43) Pub. Date: Apr. 6, 2006

(54) Viscoelastic composition, methods of use and packaging device with anti-oxidant

(75) Inventors: Erning Xia, Penfield, NY (US); George Green, Victor, NY (US)

Correspondence Address: Bausch & Lomb Incorporated, One Bausch & Lomb Place, Rochester, NY, 14604-2701, US

(73) Assignee: Bausch & Lomb Inc.

(21) Appl. No.: 11/157,295

(22) Filed: Jun. 21, 2005

Related U.S. Application Data
(60) Provisional application No. 60/614,274, filed on Sep. 29, 2004.

(51) Int. Cl. A61F 2/00; A61K 9/14; A61F 9/00; B65D 83/10

(52) U.S. Cl. 424/427; 606/107; 206/364; 424/488

(57) ABSTRACT

The present invention is directed to a viscoelastic composition comprising an aqueous solution having a viscoelastic polymer based upon the total volume of the viscoelastic composition. Typically, the viscoelastic composition further contains soluble vitamin derivative. The present invention also includes methods of use of the new viscoelastic composition and a packaging device.

### CROSS REFERENCE

This application claims the benefit of Provisional Patent Application No. 60/614,274 filed Sep. 29, 2004 and is incorporated herein by reference.

### BACKGROUND OF THE INVENTION

1. Field of the Invention

This invention relates to a viscoelastic composition, method of use and related device used in viscosurgical applications and more particularly to a viscoelastic composition used in ophthalmic surgical application such as cataract removal surgery.

2. Discussion of the Related Art

In the past decade, advances in the technology of eye surgery have made surgical treatment of eye disease and deformities attractive to alternative therapies. Cataract removal is one of the more common surgical procedures. Cataracts are opacities of the ocular lens, which generally arise in the elderly. Typically, cataract surgery involves removal of the cataractous lens from the capsular bag and replacement of the cataractous lens with a synthetic intraocular lens. Presently, this procedure involves making an incision through the sclera into the anterior chamber of the patient's eye. Another incision is made into the capsular bag. The cataractous lens is fractured in the capsular bag by procedures such as phacoemulsification and removed from the capsular bag by procedures such as aspiration. Thereafter an intraocular lens is inserted into the capsular bag and deployed therein.

The overall procedure is potentially traumatic to the capsular bag and the tissue surrounding the anterior chamber. It is advantageous to reduce the amount of trauma to any living tissue in the patient's eye during a surgical procedure. Particularly, corneal endothelial cells are sensitive to damage. Damage to the corneal endothelial cells is permanent. Serious damage can eventually lead to corneal tissue damage and loss of eyesight.

Moreover, the process of phacoemulsification produces free radicals and/or oxidants. Free radicals and/or oxidants are unstable and react somewhat indiscriminately with biological molecules in tissue. For example, a free radical and/or oxidant that are produced in phacoemulsification can damage proteins, cell walls or even the DNA of a cell. It is advantageous to reduce the damage caused by these free radicals and/or highly reactive ions.

Viscoelastic compositions are injected in the anterior chamber of the eye and the capsular bag during surgery to protect the tissue from physical trauma. The viscoelastic compositions provide a physical barrier or cushion between the instruments and the tissue. Furthermore, viscoelastic compositions maintain the shape of a cavity during operation including the anterior chamber and capsular bag. Viscoelastic compositions have been known to contain agents that are free radical scavengers and/or antioxidants.

Selection of an ingredient in a viscoelastic composition for the purpose of controlling free-radical activity and/or antioxidants, require satisfying several criteria. The ingredient cannot negatively impact the viscoelastic properties, irritate tissue or cause an adverse immune response. The ingredient should be effective as a free-radical scavenger and/or antioxidant under conditions of desirable pH and osmolality. Of course, the effectiveness of the free-radical scavenger to dampen free radical activity is an important factor.

U.S. Pat. No. 5,880,107 discloses a viscoelastic composition for use in eye surgery. The viscoelastic composition contains hyaluronic acid as the primary ingredient to provide appropriate viscoelasticity. The composition further contained a citric acid salt, typically tri-sodium citrate, an antioxidant tolerated by the intraocular tissues and a phosphate buffer. The antioxidant was selected from the group comprising glucose, sulphides, superoxide dismutase (SOD), cysteine and derivates thereof. Furthermore, other antioxidants that could be used include antioxidants, which have at least one —SH or —CHO group, peptides and enzymes.

U.S. Pat. No. 6,086,597 discloses a sodium hyaluronate viscosurgical composition that contains a compound to as a scavenger including superoxidedismutase, mannitol and glutathione. Furthermore, the use of Vitamin E (tocopherol) as an antioxidant in a viscosurgical composition is known in the art.

U.S. Pat. Nos. 5,603,929 and 5,653,972 disclose preserved, storage-stable ophthalmic compositions comprising acidic drugs, a polymeric quaternary ammonium compound and boric acid, and methods for controlling ocular inflammation using such compositions. Vitamin E tocopherol polyethylene glycol 1000 succinate is disclosed as a formulation component. U.S. Pat. No. 5,886,030 discloses the use of Vitamin E tocopherol derivatives, including Vitamin E tocopherol polyethylene glycol 1000 succinate, in anti-inflammatory ophthalmic compositions. Methods are disclosed for treating or controlling ocular inflammation and for improving comfort and reducing irritation in compositions containing ophthalmic therapeutic agents, which are irritating to the eye. The compositions may include preservatives such as benzalkonium chloride, Polyquad® and Dymed® (polyhexamethylenebiguanide).

U.S. Patent Application No. 2003-0068250 discloses the use of vitamin derivative component, including vitamin E tocopherol polyethylene glycol succinate (TPGS), with a preservative in an ophthalmic preservative composition.

While significant improvements have been made in the rheological properties of viscoelastic compositions, there still exists a need for a composition that reduces the free radical and/or oxidant activity without negatively impacting the viscoelastic properties of the viscoelastic composition. The present invention addresses these and other needs.

### SUMMARY OF THE INVENTION

The present invention is a viscoelastic composition that comprises an aqueous solution of a viscoelastic polymer and a soluble vitamin derivative. The composition is an effective viscosurgical device for use in cataract surgery to maintain the shape of the anterior chamber or the capsular bag. Typically, the viscoelastic polymer has a minimum of about 0.01% w/v and a maximum of about 20% w/v based upon the total volume of the viscoelastic composition.

The present invention also comprises a method of temporarily maintaining space in a cavity in human tissue. The method comprises the step of injecting the viscoelastic composition of one or more embodiments of the present invention into the cavity. The viscoelastic composition, then, is removed from the cavity. Preferably, the cavity is the anterior chamber of the eye or the capsular bag.

In another embodiment, there is a method of protecting tissue from trauma during a surgical procedure. The method includes coating at least a portion of the tissue with a viscoelastic composition of any one embodiment of the present invention. A surgical procedure is performed near the tissue after the coating. After the surgical procedure, at least a portion of the viscoelastic composition is removed from the tissue after the surgical procedure is performed.

In still another embodiment, there is a method of replacing a natural lens from an eye. The method comprises providing a passage through a sclera into an anterior chamber of the eye. At least a portion of the aqueous humor is removed from the anterior chamber. A viscoelastic composition according to one or more embodiments of the present invention is inserted into the anterior chamber. The corneal lens is phacoemulsified in the capsular bag. Substantially the entire lens is removed from the capsular bag. The viscoelastic composition is inserted into the capsular bag. An intraocular lens is then inserted into the capsular bag.

In another embodiment, there is a package for a viscoelastic composition, the package comprising a syringe containing a viscoelastic composition according to any embodiment, aspect, feature, combination or concept disclosed herein.

### DETAILED DESCRIPTION OF THE INVENTION

Introduction

The present invention is directed to a viscoelastic composition comprising an aqueous solution having a minimum of about 0.01% w/v and a maximum of about 20% w/v of a viscoelastic polymer based upon the total volume of the viscoelastic composition. Typically the viscoelastic composition further contains a soluble vitamin derivative. “Soluble vitamin derivative” is defined as a vitamin that has been chemically modified by adding a substituent group to the vitamin to form a derivative that has a solubility greater than the original vitamin. The present invention also includes methods of use and a device. In one embodiment, the solubility of the soluble vitamin derivative is a minimum of about 100 μg/ml, about 500 μg/ml, about 1000 μg/ml or about 5000 μg/ml.

Definitions

Viscosurgically pure as it pertains to a viscoelastic composition or ingredient thereof is defined as a level of purity that is sufficiently free of impurities to meet or exceed the United States Food and Drug Administration standards for a viscosurgical viscoelastic effective at the time this application is filed.

Polysaccharides are defined as saccharides that have 10 or more saccharide monomer units.

Zero-shear viscosity is defined as the extrapolation of the viscosity of a liquid to a zero-shear rate from measurements of viscosity as the shear rate approaches zero measured on a plate and cone rheometer at 34° C.

High-shear viscosity is defined as the viscosity of a liquid measured on a plate and cone rheometer at 34° C. with a shear rate of 300 s⁻¹.

Pseudoplastic material is defined as a material that has relatively high viscosity under low-shear and relatively low viscosity under high-shear conditions.

The phrase, “removing substantially all” as it relates to lenses and lens fragments is defined as removing a sufficient quantity that an effective implantation of an intraocular lens is not inhibited thereafter. According to one embodiment, an effective removal of the lens requires a minimum of 90% w/v of the lens, 95% w/v of the lens or 98% w/v of the lens.

A cannula is defined as any tubular member having a passage that is configured to penetrate tissue and deliver a device through the passage.

The percentage of quenching as describe in the application with the exception of the examples is defined as the percentage amount that free-radical activity is prevented as evaluated by the 2-deoxy-D-ribose (2-DR) oxidation method. This is a conventional method of OH-radical detection forming by the Fenton reaction, radiation or ultrasound. It is based on its reaction with 2-DR. the obtained product of degradation, after a thermoactivated reaction with thiobarbituric acid (TBA), produces a pink chromogen quantified by HPLC.

Formulation

According to one embodiment of the present invention, there is a viscoelastic composition comprising an aqueous solution having a minimum of about 0.01% w/v and a maximum of about 20% w/v of a viscoelastic polymer based upon the total volume of the viscoelastic composition. Typically the viscoelastic composition further contains soluble vitamin derivative.

As used herein, a vitamin derivative component or vitamin-based surfactant component includes a vitamin derivative with a chemical structure such that the resulting moiety is effective as a surfactant in the present compositions and/or methods. Without wishing to limit the invention to any particular theory of operation, it is believed that the vitamin derivative components of the present invention form micelles and have a critical micelle concentration. Such components can at least assist in cleaning contact lenses in or out of the eye, can at least assist in removing, preferably can remove foreign particulates, debris, antigens and the like from the eye and are ophthalmically acceptable and/or substantially non-toxic to the eye.

Vitamin-based surfactants in accordance with the present invention can be used alone or in combination or mixtures with one another. Vitamins A, A2, C, D1, D2, D3, D4, E, K1, K2 and folic acid are preferred vitamins, which can be derivatized to form surfactants in accordance with the present invention. Any suitable vitamin derivative can be employed provided such derivative is effective to function as described herein, for example, as a surfactant. Examples of such derivatives include, but not limited to, the succinic acid ester or amide of Vitamin A (retinol), which can be prepared and then further esterified or amidated (e.g., coupled with an amide bond) with a polyalkylene glycol, for example, selected from polyethylene glycols, polypropylene glycols, mixtures thereof and the like, to form a suitable surfactant. Polyethylene glycol 1000 is one very useful polyalkylene glycol. Vitamins D1, D2, D3 or D4 can be similarly esterified or amidated to form suitable surfactants. The dihydro derivatives of Vitamins K1 and K2 can be prepared and thereafter esterified or amidated with succinic acid and polyalkylene glycol, e.g., polyethylene glycol 1000. Folic acid can be esterified or amidated at either of its two free carboxylic acid groups by polyalkylene glycol, e.g., polyethylene glycol 1000. Non-anionic Vitamin E derivatives are particularly preferred.

Two very useful Vitamin E derivative-based surfactants are D-α-tocopherol polyethylene glycol 1000 succinate (Vitamin E TPGS), and its amide analogue (Vitamin E TPGSA). The structure of Vitamin E TPGS is represented as follows:

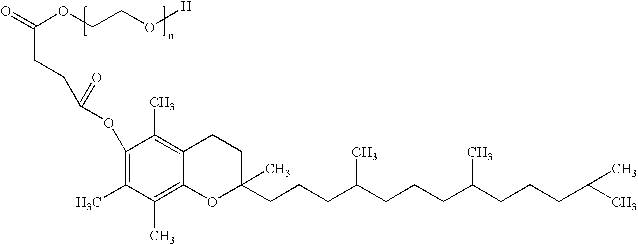

Vitamin E TPGS is a polyethylene glycol (PEG) ester of D-α-tocopherol acid succinate, where the polyethylene glycol (PEG) molecular weight is about 1000. D-α-tocopherol acid succinate is, in turn, a succinic acid ester of D-α-tocopherol, Vitamin E. Vitamin E TPGSA is a polyethylene glycol (PEG) amide of D-α-tocopherol acid succinate, containing an amide bond between the PEG chain and the distal succinic acid free acid group, and where the PEG molecular weight is about 1000. The structure of TPGSA is represented as follows.

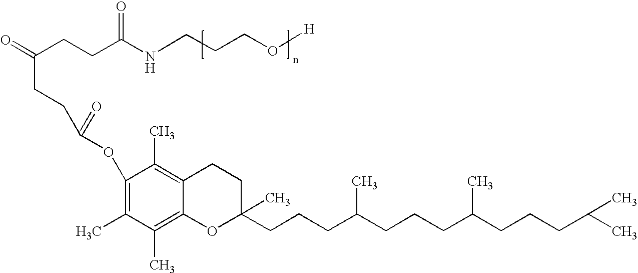

Other Vitamin E-based surfactant components can also be used in the present invention. All of the naturally occurring Vitamin E compounds that exhibit at least part of the biological activity of α-tocopherol and their corresponding synthetic forms can be used to form surfactants for use in the present invention. Vitamin E compounds useful for the present invention include, without limitation, α-, β-, χ-, and δ-tocopherol and α-, β-, χ-, and δ-tocotrienol. All these compounds occur as a variety of isomers. The commercially available synthetic forms of Vitamin E comprise an approximately equal mixture of eight sterioisomeric forms of α-tocopherol. The presently useful surfactants can be based on a single vitamin isomer or a mixture of vitamin isomers.

A variety of surfactant forms of vitamins such as Vitamin E or other vitamins, can be produced and are useful in the present invention. These surfactants can be produced with known synthetic chemistry methodology, for example, direct esterification or amidation of the phenolic OH group of tocopherol with a surfactant chain such as polyethylene glycol or a second esterification or amidation of a primary-ester intermediate such as Vitamin E succinate. Esterification of the phenolic OH group of tocopherol is a preferred chemical synthetic path. The vitamin-based esters are preferred for use as surfactants in accordance with the present invention. Ester or amide bonds are readily hydrolyzed by enzymes, such as enzymes, with esterase or amidose activity in-vivo, which leads directly to the production of biologically active vitamins. Ocular tissues are known to contain esterases and amidoses, which hydrolyze ester and amide bonds, respectively in ocular prodrugs. Thus, ocular tissue esterases or amidoses can act upon the ester or amide bonds in, for example, Vitamin E TPGS and other ester-based vitamin surfactants to release biologically active vitamins to tissues.

Vitamin-based surfactants, which have ester structures, are often sufficiently stable to substantially and effectively maintain surfactant activity in the aqueous compositions, e.g., solutions, of the present invention. Other types of surfactants can be produced with other in-vivo labile bonds, which also lead to the production of biologically active vitamins, provided that such surfactants have sufficient stability in aqueous solution to provide an acceptable level of surfactant activity. Amide bonds, as an example, are estimated to be several orders of magnitude more hydrolytically stable than ester bonds. Nonionic surfactants are preferred. Under certain conditions, for example, where the activity of the disinfecting agent is not compromised and in-eye safety and comfort can be maintained, cationic, amphoteric and anionic surfactants can also be employed. A variety of nonionic surfactant classes may be produced based on a vitamin precursor and used in the present invention, such as the polyethylene glycol surfactants. Other nonionic surfactant classes corresponding to the presently useful vitamin-based surfactant components include, without limitation, the polyoxyethylated linear alcohols, nonoxynols, octoxynols, polyoxyethylated dodecylamines, sorbitan monoesters and the like and mixtures thereof.

The surfactants useful in the present invention advantageously are water-soluble when used alone or as a mixture. Such surfactants preferably have HLB values of about 12 to about 13 when used alone. In addition, the surfactant or surfactants employed preferably produce a clear solution in accordance with the present invention.

Vitamin E TPGS is known to be unstable with respect to hydrolysis upon exposure to acidic and alkaline pH conditions. The instability is due to acid- or base-catalyzed hydrolysis of the ester linkages. As pH approaches neutrality in buffered solutions, Vitamin E TPGS becomes more stable. This can be problematic for some compositions, as pH 7.5 is more optimal for ocular comfort, since it is closer to the human tear pH of 7. 45. Therefore, compositions of the present invention can also optionally include the hydrolysis products of Vitamin E surfactant ester bond hydrolysis, to further stabilize the surfactant ester against hydrolysis during shelf storage. Hydrolysis products of ester forms of other vitamin-based surfactants can also be used in a similar manner to stabilize the surfactant as well as provide an additional source of vitamin.

Hydrolysis of Vitamin E TPGS to Vitamin E tocopherol hemisuccinate and polyethylene glycol can result in a solution, which is incompatible with disinfecting agents such as polyhexamethylene biguanide (PHMB). This is because it has been found that in some cases the amount of Vitamin E tocopherol hemisuccinate anion which forms can substantially ion-pair the cationic PHMB, thus reducing or neutralizing PHMB antimicrobial activity. Hydrolysis of Vitamin E TPGS in aqueous solution is quite slow and in most cases results in part-per-million concentrations of hydrolysis products. Nonetheless, this may be sufficient to inactivate a disinfecting agent such as PHMB or other cationic antimicrobial agents. In such cases, an alternative amide-based surfactant, such as Vitamin E TPGSA, may be advantageously employed. It may be useful to suppress hydrolysis of the Vitamin E TPGS. Such suppression may be achieved by employing small amounts of polyethylene glycol, for example, PEG 1000, and succinic acid, buffering the solution at a slightly acidic pH of about 6.8-6.9, and using a sterically-hindered, non-complexing buffer, such as bis tris, 2-(bis(2-hydroxyethyl)amino)-2(hydroxymethyl)-1,3-propanediol. The relatively bulky hydrophilic hydroxyethyl groups of bis tris act to shield the basic nitrogen and make it more difficult for this buffer to serve as a base-catalyst for ester hydrolysis. It is preferred to use more stable amide-based vitamin surfactant in compositions containing conventional amounts of PHMB (for example, about 0.5 ppm to about 1 ppm) where such compositions may otherwise result in unstable Vitamin E TPGS.

The vitamin-based surfactants of the present invention can also be used together with conventional non-vitamin surfactants, for example, nonionic, cationic, anionic and amphoteric non-vitamin surfactants. The vitamin-based surfactants, if used in combination with one or more non-vitamin surfactants are preferably used with nonionic non-vitamin surfactants.

In one embodiment, the viscoelastic composition has a concentration of soluble vitamin derivative that is a minimum of about 0.01% w/v and/or a minimum of about 4% w/v based upon the total weight of the viscoelastic composition. Typically, the concentration of soluble vitamin derivative is a minimum of about 0.2% w/v and a maximum of about 3% w/v based upon the total volume of the viscoelastic composition. Preferably, the concentration of soluble vitamin derivative is a minimum of about 0.3% w/v, about 0.4% w/v or about 0.5% w/v and a maximum of about 2% w/v, about 1% w/v or about 5% w/v based upon the volume of the viscoelastic composition in one aspect of the invention.

The viscoelastic composition of one embodiment of the present invention has a ratio of the viscosity of the viscoelastic composition to the viscosity of a comparable viscoelastic composition having no soluble vitamin derivative that is a minimum of about 1 and a maximum of about 2.5. A comparable viscoelastic composition is defined as a viscoelastic composition that has all of the same chemical ingredients as the viscoelastic composition at the same concentrations except it has soluble vitamin derivative. Typically, the ratio of the viscosity of the viscoelastic composition to the viscosity of a comparable viscoelastic composition is a minimum of about 1, about 1.1 and about 1.2 and a maximum of about 2.5, about 2.2 and about 2.

The viscoelastic composition of yet another embodiment quenches chemical scavengers effectively, wherein the percentage of quenching is a minimum of about 45%. Typically, the percentage quenching is greater than about 55%, about 65%, about 70%, about 75% or about 80% according to the method of testing known in the art.

The viscoelastic composition comprises one or more viscoelastic polymers that are useful and known as viscosurgical devices. In one embodiment, the viscoelastic polymer is selected from the group comprising hyaluronic acid, hydroxypropylmethylcellulose, polyacrilyc acid, carbopol, polyvinylalchol, polyvinylpirrolidone, condroitin sulfate, polycarbophil, methylcellulose, carboxymethylcellulose, hydroxyethylcellulose, hydroxypropylcellulose, ethylcellulose, polyethylene oxides, alginate, pectin, xanthan gum, dextrans, collagen and derivatives threof and salts thereof and combinations thereof.

In one embodiment, the average molecular weight of the viscoelastic polymer, including a polysaccharide, is a minimum of about 20 kD and a maximum of about 5,000 kD. Generally, the average molecular weight of a viscoelastic polymer, including polysaccharide, is a minimum of about 30 kD, about 50 kD, about 70 kD, about 400 kD, about 500 kD, about 750 kD or about 1,000 kD. Typically, the average molecular weight of a viscoelastic polymer, including a polysaccharide, is a maximum of about 50 kD, about 80 kD, about 100 kD, about 200 kD, about 400 kD, about 500 kD, about 1,000 kD or about 3,000 kD.

Typically, there are two general classes of viscoelastic compositions. A dispersive viscoelastic composition has properties that disperse or coat the tissue well and adhere well to the tissue. A dispersive viscoelastic composition (also known as an “adhesive viscoelastic composition”) typically has a low molecular weight. A cohesive viscoelastic composition is better at maintaining the space in a cavity in human tissue and is less likely to leak from the cavity under low or zero shear conditions. Typically, a cohesive viscoelastic composition has a high molecular weight.

In one embodiment, the average molecular weight of a viscoelastic polymer in a dispersive viscoelastic composition is a minimum of about 20 kD, 30 kD, about 50 kD or about 70 kD. Typically, the average molecular weight of a viscoelastic polymer in a dispersive viscoelastic composition is a maximum of about 50 kD, about 80 kD, about 100 kD, about 200 kD, about 400 kD or about 500 kD.

In another embodiment, the average molecular weight of a viscoelastic polymer in a cohesive viscoelastic composition is a minimum of about 400 kD, about 500 kD, about 750 kD or about 1,000 kD. Typically, the average molecular weight of a viscoelastic polymer in a cohesive viscoelastic composition is a maximum of about 1,000 kD, 3,000 kD or about 5,000 kD.

The concentration of the viscoelastic polymer is a minimum amount of about 0.01% w/v and a maximum amount of about 20% w/v based upon the total weight of the viscoelastic composition in one embodiment. Typically, the concentration of the viscoelastic polymer is a minimum of about 0.1% w/v, about 0.2% w/v, about 1.0 or about 2.0% w/v and a maximum of about 0.3% w/v, about 0.5% w/v, about 1% w/v, about 2% w/v about 3% w/v, about 5% w/v or about 15% w/v based upon the total weight of the viscoelastic composition.

In still another embodiment, the viscoelastic polymer comprises a mixture of hyaluronic acid and/or salts thereof and hydroxypropylmethylcellulose.

The concentration of hyaluronic acid and/or salts thereof is a minimum of about 0.1% w/v and a maximum of about 6% w/v based upon the volume of the viscoelastic composition in one embodiment. Typically, the concentration of hyaluronic acid and/or salts thereof is a minimum of about 0.3% w/v, about 0.6% w/v or about 1% w/v and a maximum of about 6% w/v, about 4% w/v or about 2% w/v based upon the volume of the viscoelastic composition.

The average molecular weight of the hyaluronic acid and/or salts thereof is a minimum of about 500 kD and a maximum of about 5000 kD in one embodiment. Typically, the average molecular weight of the hyaluronic acid and/or salts thereof is a minimum of about 500 kD, about 700 kD or about 1000 kD and a maximum of about 4000 kD, about 3000 kD or about 2000 kD.

The concentration of hydroxypropylmethylcellulose is a minimum of about 0.05% w/v and a maximum of about 5% w/v based upon the volume of the viscoelastic composition in one embodiment. Typically, the concentration of hydroxypropylmethylcellulose is a minimum of about 0.2% w/v, about 0.4% w/v or about 0.8% w/v and a maximum of about 5% w/v, about 3% w/v or about 1% w/v based upon the volume of the viscoelastic composition.

The average molecular weight of the hydroxypropylmethylcellulose is a minimum of about 10 kD and a maximum of about 120 kD according to one embodiment. Typically, the average molecular weight of the hydroxypropylmethylcellulose is minimum of about 10 kD, about 12 kD or about 20 kD and a maximum of about 120 kD, about 90 kD or about 86 kD.

In one embodiment, the viscoelastic polymer comprises a polysaccharide. In another embodiment, the viscoelastic polymer is preferably a polysaccharide selected from the group comprising hyaluronic acid, hydroxypropylmethylcellulose, condroitin sulfate, methylcellulose, carboxymethylcellulose, hydroxyethylcellulose, hydroxypropylcellulose, ethylcellulose, alginate, pectin, dextrans, collagen, proteoglycans, polyvinylpyrrolidone, keratin carrageenans and derivatives thereof and salts thereof and combinations thereof. Alternatively, the viscoelastic is a synthetic polymer selected from the group comprising polyacrylamide and poly (n-dimethyl)acrylamide.

The viscoelastic polymer comprises alginate in one embodiment. Typically the concentration of alginate is a minimum of about 0.05% w/v and a maximum of about 9% w/v based upon the volume of the viscoelastic composition. Optionally, the minimum alginate concentration is about 1% w/v, about 1.5% w/v, about 2% w/v, about 3% w/v or about 4% w/v based upon the total weight of the viscoelastic composition. Optionally, the maximum alginate concentration is about 10% w/v, about 8% w/v, about 6% w/v, about 4% w/v, about 3% w/v or about 2% w/v based upon the total weight of the viscoelastic composition. Preferably, the alginate concentration is a minimum of about 2% w/v and a maximum of about 5.25% w/v.

In one embodiment, the average molecular weight of the alginate is a minimum of about 50 kD and a maximum of about 5,000 kD. Typically, the average molecular weight of the alginate is a minimum of about 100 kD, about 200 kD, about 500 kD or about 1000 kD. Typically, the average molecular weight of the alginate is a maximum of about 2000 kD, about 1000 kD, about 750 kD or about 500 kD.

The viscoelastic composition has one or more properties including but not limited to osmolality, pH, zero-shear viscosity and high-shear viscosity. The osmolality of the viscoelastic composition is a minimum of about 200 mOsmol/Kg and a maximum of about 400 mOsmol/Kg in an embodiment. Typically, the osmolality of the viscoelastic composition is a minimum of about 220 mOsmol/Kg, about 260 mOsmol/Kg, about 280 mOsmol/Kg, about 300 mOsmol/Kg or about 320 mOsmol/Kg and a maximum of about 400 mOsmol/Kg, about 380 mOsmol/Kg, about 360 mOsmol/Kg or about 340 mOsmol/Kg.

The zero-shear viscosity of the viscoelastic composition is a minimum of about 6·10⁴ cps and a maximum of about 4·10⁶ cps. Generally, the zero-shear viscosity of the viscoelastic composition is a minimum of about 6×10⁴ cps, about 4×10⁵ cps or about 8×10⁵ cps and a maximum of about 3.5×10⁶ cps, about 1.8×10⁶ cps or about 1.2×10⁶ cps.

The high-shear viscosity of the viscoelastic composition is a minimum of about 500 cps and a maximum of about 2000 cps. Generally, the high-shear viscosity of the viscoelastic composition is a minimum of about 500 cps, about 600 cps or about 700 cps and a maximum of about 2000 cps, about 1500 cps or about 1000 cps.

The pH of the viscoelastic composition of one embodiment is a minimum of about 5 and a maximum of about 8. In one embodiment, the pH of the viscoelastic composition is a minimum of about 5.5, about 6 or about 6.5 and a maximum of about 7.5, about 7.2 or about 7.

The viscoelastic composition of one embodiment has a formulation set forth in Table 1.

TABLE 1

| Component or Property of the | |
|---|---|
| Viscoelastic Composition | Amount |
| 1.0 × 10⁶-3 × 10⁶ Molecular Weight | 0.5% w/v to 3% w/v |
| Hyaluronic Acid or Salt Form Thereof | |
| 20,000-200,000 Molecular Weight Hydroxy- | 0.1% w/v to 2% w/v |
| propylmethylcellulose | |
| Vitamin E TPGS | 0.1% w/v to 4% w/v |
| Buffered to pH with a phosphate buffer system | 6.9 to 7.5 |
| Osmolality adjusted to | 290-350 mOsm/l |

In one preferred embodiment the viscoelastic composition comprises the following:
2% w/v hyaluronic acid (MW 1.8×10⁶)
0.5% w/v hydroxypropylmethylcellulose (MW 86,000)
1% w/v vitamin E TPGS
purified water q. s. to 100 ml
pH 7.3
335 mOsm/l

In another preferred embodiment, the viscoelastic composition comprises the following:
2% w/v hyaluronic acid (MW 1.8×10⁶)
0.8% w/v hydroxypropylmethylcellulose (MW 86,000)
1% w/v vitamin E TPGS purified water q. s. to 100 ml
pH 7.3
335 mOsm/l

In another preferred embodiment, the viscoelastic composition comprises the following:
2% w/v hyaluronic acid (MW 1.8×10⁶)
1% w/v hydroxypropylmethylcellulose (MW 86,000)
1% w/v vitamin E TPGS purified water q. s. to 100 ml
pH 7.3
335 mOsm/l

In another preferred embodiment, the viscoelastic composition comprises the following:
5.25% w/v alginate
1% w/v vitamin E TPGS
Purified water q. s. a. d. to 100% w/v
   Methods of Use

Viscoelastic composition according to any one or more of the foregoing embodiments, concepts or aspects including combinations and variations of the foregoing embodiments can be used according to the following method or methods.

In one embodiment, there is a method of maintaining space in a cavity in human tissue. The method comprises the step of injecting, into the cavity, a viscoelastic composition according to any embodiment, aspect, feature, combination or concept disclosed herein. Thereafter, the viscoelastic composition is removed from the cavity. Preferably, the cavity is the anterior chamber of the eye or the capsular bag.

In still another embodiment, there is a method of protecting tissue from trauma during a surgical procedure. The method comprises the step of coating at least a portion of the tissue with a viscoelastic composition according to any embodiment, aspect, feature, combination or concept disclosed herein. Preferably, the tissue that is covered is in the anterior chamber of the eye and/or the capsular bag. A surgical procedure is then performed near the tissue. When the surgical procedure is completed, at least a portion of the viscoelastic composition is removed from the tissue.

In one embodiment, there is a method of replacing a natural lens from an eye. Examples of procedures for removing a lens from a patient's eye include but are not limited to U.S. Pat. No. 3,589,363 (cataract surgery), U.S. Pat. No. 3,693,613 (phacoemulsification) and U.S. Pat. No. 5,718,676 (process using micro flow needle), which are all incorporated herein by reference in their entirety. The process generally includes providing a passage through a sclera or cornea into an anterior chamber of the eye. The process involves making a small incision into the sclera or cornea. Alternatively or additionally, a cannula or trochar is used to create a passage through the sclera or cornea. Preferably, the incision or passage is as small as possible. Preferably, the incision or passage is smaller than about 5 mm, about 4 mm or about 3 mm. Thereafter, the aqueous humor is withdrawn or otherwise removed from the anterior chamber of the eye.

A viscoelastic composition according to any one of the embodiments, aspects concepts, combinations or features is inserted into the anterior chamber. The viscoelastic composition maintains the space in the anterior chamber. The viscoelastic composition coats the tissue in the wall of the anterior chamber.

According to one embodiment, there is a package for a viscoelastic composition that includes a delivery device. The device delivers a viscoelastic composition into the anterior chamber of a patient's eye. The device includes a syringe that contains a viscoelastic composition according to any embodiment, aspect, combination, concept or feature disclosed herein.

The syringe further comprises an outlet port and, optionally, a cannula configured to sealably connect to the outlet port. The cannula has a maximum inner diameter of about 2 mm. Typically, the maximum inner diameter is about 1.8 mm, about 1.5 mm or about 1 mm. Generally, the minimum inner diameter is about 0.8 mm, about 0.6 mm or about 0.4 mm.

In one embodiment, the viscoelastic composition requires a maximum force of 30 N to pass through a stainless steel cannula having a length of 2.2 cm and an inner diameter of 0.5 mm at a delivery rate of 0.02 ml/sec. Preferably, the viscoelastic composition requires a maximum force of about 27 N, about 25 N, about 20 N or about 18 N to pass through a stainless steel cannula having a length of 2.2 cm and an inner diameter of 0.5 mm at a delivery rate of 0.02 ml/sec.

Once the viscoelastic composition is inserted into the anterior chamber the crystalline lens is removed. The technique for removing the lens includes performing a capsulorhexis incision and breaking down the lens into smaller pieces through phacoemulsification or other known techniques. Thereafter, the pieces are removed by, for example, aspiration.

The viscoelastic composition is inserted into the capsular bag for space maintenance purposes. Moreover, the viscoelastic composition coats the capsular bag and protects it for additional steps in the surgical procedure.

According to one embodiment, the intraocular lens is inserted into the capsular bag. Typically, there is a method of inserting an intraocular lens into a capsular bag of an eye. The method comprises providing a lens insertion device comprising a loadable chamber configured to receive the intraocular lens, a tapered conduit having a first end connected to the loadable chamber and a second end. The second end is configured to penetrate through the passage in the corneal lens and into the capsular bag. An example of a lens insertion device is found in U.S. Pat. No. 6,558,419, which is incorporated herein by reference in its entirety. The lens insertion device is further configured with a slidable actuator. The slidable actuator of one embodiment is configured to actuate the intraocular lens through the conduit past the second end. Typically, the second end of the tapered conduit has an inner diameter that is a maximum of about 5 mm. Preferably the second end of the tapered conduit has an inner diameter that is a maximum of about 4 mm about 3.5 mm, about 3 mm or about 2.8 mm. Preferably, a maximum force of about 30 N is required to deliver the intraocular lens through the cannula. More preferably, a maximum force of about 27 N, about 25 N, about 20 N or about 18 N is required to deliver the intraocular lens through the cannula.

Prior to deployment, at least a portion of the intraocular lens is coated with a viscoelastic composition according to any one of the embodiments, aspects, concepts, combinations or features of the present invention. The intraocular lens is loaded into the loadable chamber either before or after it is coated. The conduit is inserted through the passage. The actuator forces the intraocular lens through the passage and into the capsular bag. After the intraocular lens is deployed, the conduit is removed from the passage.

Typically, at least a portion of the viscoelastic composition is removed from the capsular bag and/or anterior chamber. A physiological solution is then used to fill the anterior chamber. The sclera and/or cornea are sutured to close the passage.

Although preferred embodiments have been depicted and described in detail, it will be apparent to those skilled in the relevant art that the specification has been prepared without the intention of limiting the scope of the invention and that various modifications, additions, substitutions, and the like can be made without departing from the spirit of the invention and these are therefore considered to be within the scope of the invention as defined in the claims which follow.

What is claimed is:

1. A viscoelastic composition comprising an aqueous solution having a minimum of about 0.01% w/v and a maximum of about 20% w/v of a viscoelastic polymer based upon the total volume of the viscoelastic composition and further having soluble vitamin derivative.

2. The composition of claim 1, wherein the concentration of soluble vitamin derivative is a minimum of about 0.1% w/v and a maximum of about 4% w/v based upon the total weight of the viscoelastic composition.

3. The composition of claim 1, wherein the soluble vitamin derivative includes one or more derivatives of a vitamin selected from the group consisting of Vitamin A, Vitamin A2, Vitamin C, Vitamin D1, Vitamin D2, Vitamin D3, Vitamin D4, Vitamin E, Vitamin K1, Vitamin K2, folic acid and mixtures thereof.

4. The composition of claim 1, wherein the soluble vitamin derivative is present in the viscoelastic in a minimum amount of 0.2% w/v and a maximum amount of 3% w/v.

5. The composition of claim 1, wherein the soluble vitamin derivative includes at least one non-anionic derivative of Vitamin E.

6. The composition of claim 1, wherein the soluble vitamin derivative component includes one or more amides chemically linked to a vitamin.

7. The composition of claim 1, wherein the soluble vitamin derivative includes Vitamin E TPGS or Vitamin E TPGSA.

8. The composition of claim 7, wherein the soluble vitamin derivative is present in the viscoelastic composition in a minimum amount of 0.1% w/v and a maximum amount of 4% w/v.

9. The composition of claim 1, wherein the soluble vitamin derivative has a water solubility greater than 100 μg/ml.

10. The composition of claim 1, wherein the ratio of the viscosity of the viscoelastic composition to the viscosity of a comparable viscoelastic composition having no soluble vitamin derivative is a minimum of about 1 and a maximum of about 2.5.

11. The composition of claim 1, wherein the percentage of quenching is a minimum of about 45%.

12. The composition of claim 1, wherein the viscoelastic polymer is selected from the group comprising hyaluronic acid, hydroxypropylmethylcellulose, polyacrilyc acid, carbopol, polyvinylalchol, polyvinylpirrolidone, condroitin sulfate, polycarbophil, methylcellulose, carboxymethylcellulose, hydroxyethylcellulose, hydroxypropylcellulose, ethylcellulose, polyethylene oxides, alginate, pectin, xanthan gum, dextrans, collagen and derivatives thereof and salts thereof and combinations thereof.

13. The composition of claim 1, wherein the viscoelastic polymer comprises a polysaccharide.

14. The composition of claim 13, wherein the polysaccharide is selected from the group comprising hyaluronic acid, hydroxypropylmethylcellulose, condroitin sulfate, methylcellulose, carboxymethylcellulose, hydroxyethylcellulose, hydroxypropylcellulose, ethylcellulose, alginate, pectin, dextrans, collagen, proteoglycans, polyvinylpyrrolidone, keratin carrageenans and derivatives thereof and salts thereof and combinations thereof.

15. The composition of claim 1, wherein the viscoelastic polymer comprises alginate.

16. The composition of claim 15, wherein the concentration of alginate is a minimum of about 0.05% w/v and a maximum of about 9% w/v based upon the volume of the viscoelastic composition.

17. The composition of claim 15, wherein the average molecular weight of the alginate composition of yet minimum of about 50 kD and a maximum of about 5,000 kD.

18. The composition of claim 1, wherein the viscoelastic polymer comprises a mixture of hyaluronic acid and/or salts thereof and hydroxypropylmethylcellulose.

19. The composition of claim 18, wherein the concentration of hyaluronic acid and/or salts thereof is a minimum of about 0.1% w/v and a maximum of about 6% w/v based upon the volume of the viscoelastic composition.

20. The composition of claim 19, wherein the average molecular weight of the hyaluronic acid and/or salts thereof composition of yet minimum of about 500 kD and a maximum of about 5000 kD.

21. The composition of claim 18, wherein the concentration of hydroxypropylmethylcellulose is a minimum of about 0.05% w/v and a maximum of about 5% w/v based upon the volume of the viscoelastic composition.

22. The composition of claim 21, wherein the average molecular weight of the hydroxypropylmethylcellulose composition of yet minimum of about 10 kD and a maximum of about 120 kD.

23. The composition of claim 1, wherein the average molecular weight of the viscoelastic polymer is a minimum of about 20 kD and a maximum of about 5,000 kD.

24. The composition of claim 1, wherein the concentration of the viscoelastic polymer is a minimum amount of about 0.01% w/v and a maximum amount of about 20% w/v based upon the total weight of the viscoelastic composition.

25. The composition of claim 1, wherein the osmolality of the viscoelastic composition is a minimum of about 200 mOsmol/Kg and a maximum of about 400 mOsmol/Kg.

26. The composition of claim 1, wherein the zero-shear viscosity of the viscoelastic composition is a minimum of about 6·10⁴ cps and a maximum of about 4·10⁶ cps.

27. The composition of claim 1, wherein the high-shear viscosity of the viscoelastic composition is a minimum of about 500 cps and a maximum of about 2000 cps.

28. The composition of claim 1, wherein the pH of the viscoelastic composition is a minimum of about 5 and a maximum of about 8.

29. A method of temporarily maintaining space in a cavity in human tissue, the method comprising the steps of:
(a) injecting the viscoelastic composition of claim 1 into the cavity; and
(b) removing the viscoelastic composition from the cavity.

30. The process of claim 29, wherein the cavity is the anterior chamber of the eye or the capsular bag.

31. A method of protecting tissue from trauma during a surgical procedure, the method comprising the steps of:
(a) coating at least a portion of the tissue with the viscoelastic composition of claim 1;
(b) performing a surgical procedure near the tissue after the step of (a) coating; and
(c) removing at least a portion of the viscoelastic composition from the tissue after the step of (b) performing.

32. The method of claim 31, wherein the step of (a) coating covers at least a portion of the tissue in an anterior chamber of an eye.

33. The method of claim 31, wherein the step of (a) coating covers at least a portion of the tissue in a capsular bag of an eye.

34. A method of replacing a natural lens from an eye, the method comprising the steps of:
(a) providing a passage through a sclera into an anterior chamber of the eye;
(b) removing at least a portion of the aqueous humor from the anterior chamber;
(c) inserting the viscoelastic composition of claim 1 into the anterior chamber;
(d) phacoemulsifying a lens in the capsular bag of the eye;
(e) removing substantially all of the lens from the capsular bag;
(f) injecting the viscoelastic composition into the capsular bag; and
(g) inserting an intraocular lens into the capsular bag.

35. The method of claim 34, further comprising the step of (b) removing at least a portion of the viscoelastic composition from the capsular bag.

36. The method of claim 34, further comprising the step of (b) removing at least a portion of the viscoelastic composition from the anterior chamber.

37. The method of claim 36, further comprising the step of suturing the sclera after the step of (g) inserting an intraocular lens.

38. The method of claim 34, wherein the step of inserting comprises coating the intraocular lens with the viscoelastic composition and delivering the intraocular lens through a cannula.

39. The method of claim 38, wherein the cannula has a tip configured to be inserted into the capsular bag, wherein the tip of the cannula has an inner diameter that is a maximum of about 1 mm.

40. The method of claim 38, wherein the step of delivering the intraocular lens through the cannula requires a maximum force of about 30 N.

41. A package for a viscoelastic composition, the package comprising a syringe containing the viscoelastic composition of claim 1.

42. The package of claim 41, wherein the syringe has an outlet port, the package further comprises a cannula configured to sealably connect to the outlet port having a maximum inner diameter of about 2 mm.

43. The package of claim 41, wherein viscoelastic composition requires a maximum force of 30 N to pass through the cannula.
